(19) United States
(12) Patent Application Publication
(10) Pub. No.: US 2023/0048252 A1
(43) Pub. Date: Feb. 16, 2023

(54) METHODS AND SYSTEMS FOR TREATMENT GUIDELINE DISPLAY

(71) Applicant: GE Precision Healthcare LLC, Wauwatosa, WI (US)

(72) Inventors: Sanand Sasidharan, Bengaluru (IN); Ravi Bhardwaj, Bengaluru (IN); Anuradha Kanamarlapudi, Bengaluru (IN); Sundararaj Mani, Brookfield, WI (US)

(21) Appl. No.: 17/819,288

(22) Filed: Aug. 11, 2022

(30) Foreign Application Priority Data
Aug. 13, 2021 (IN) 202141036688
Aug. 13, 2021 (IN) 202141036711

(51) Int. Cl. G16H 20/00

(52) U.S. Cl. CPC G16H 20/00

(57) ABSTRACT

Various methods and systems are provided for display of recommended treatment based on a patient's medical history and standardized guidelines. In one example, a computing system includes a display screen configured to display a patient medical path listing one or more of treatment guidelines and patient medical history, and to display abbreviated representations of at least one of the treatment guidelines and the patient medical history that can be reached directly from the displayed patient medical path. The abbreviated representations each display a limited list of data that is selectable to launch the treatment guidelines and/or the patient medical history and enable the selected data to be seen.

### CROSS REFERENCE TO RELATED APPLICATIONS

The present application claims priority to Indian Provisional Application No. 202141036711 entitled “METHODS AND SYSTEMS FOR TREATMENT GUIDELINE DISPLAY”, filed on Aug. 13, 2021, and to Indian Provisional Application No. 202141036688 entitled “METHODS AND SYSTEMS FOR PATIENT HISTORY SUMMARIES”, filed on Aug. 13, 2021. The entire contents of the above-identified applications are hereby incorporated by reference for all purposes.

### FIELD

Embodiments of the subject matter disclosed herein relate to analysis of patient information, and more particularly to patient history analysis and display of treatment guidelines.

### BACKGROUND

Digital collection, processing, storage, and retrieval of patient medical records may include a conglomeration of large quantities of data configured in a wide variety of formats. In some examples, the data may include numerous medical procedures and records generated during investigations of the patient, including a variety of examinations, such as blood tests, urine tests, pathology reports, image-based scans, etc. For a large hospital facility, retrieving all the relevant information stored in different formats and presenting it to a medical practitioner in an efficient, streamlined manner for analysis is challenging.

Duration of the diagnosis of a medical condition of a subject followed by treatment may be spread over time from a few days to a few months or even years in the case of chronic disease, e.g., diseases that take more than one year to cure. Examples of chronic disease include Alzheimer's disease, cancer, asthma, and diabetes, amongst others. Over the course of diagnosing and treating chronic disease, the patient may undergo many different treatments and procedures, and may move to different hospitals and/or geographic locations. Maintaining the patients records and evaluating ongoing and future directions for treatment may be a complicated task.

Clinical standardization of patient care, with respect to chronic disease, may reduce costs and increase a quality of treatment. For example, by referring to standardized cancer treatment guidelines and pathways, physicians may be better equipped to select optimized treatment routes for the patient. Use of the standardized guidelines, however, may depend on both accurate tracking of the patient's medical history and adherence to the recommendations provided by the guidelines which may be challenging due to a complexity of data and an amount of data to process.

Physicians are increasingly relying on Electronic Health Record (EHR) systems to review historical health records of the patient during diagnosis. For patients with chronic illnesses, there are often hundreds of EHRs resulting from numerous routine visits. Sorting and extracting information from past EHRs for such patients is a slow and inefficient process, increasing a likelihood of missing records with relevant data which may be spread out across a large number of less informative routine visit records. Furthermore, the standardized treatment guidelines may include numerous options at junctions along the treatment route and selecting the most suitable choice based on patient history may be difficult. As a result, patient care may deviate from the treatment guidelines, leading to adverse effects on an accuracy of diagnosis and quality of treatment.

Systems and methods are desired for matching a diagnosis of a patient to a relevant section of care guidelines and presenting the relevant guidelines to a clinician for efficient selection of treatment.

### BRIEF DESCRIPTION

In one embodiment, a computing system includes a display screen configured to display a patient medical path listing one or more of treatment guidelines and patient medical history, and additionally being configured to display abbreviated representations of at least one of the treatment guidelines and the patient medical history that can be reached directly from the displayed patient medical path. The abbreviated representations each display a limited list of data offered within the treatment guidelines and/or the patient medical history, each of the data in the list being selectable to launch the treatment guidelines and/or the patient medical history and enable the selected data to be seen. Furthermore, the abbreviated representations are displayed while in an unlaunched state. In this way, a duration of time between treatments and/or appointments may be reduced, high quality patient care throughput and patient satisfaction may be increased, and continuity of distributed care is enhanced. Furthermore, more accurate diagnosis and treatments may be provided, leading to more successful patient treatment outcomes as well as reductions in costs.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a system for displaying a patient event timeline overlaid with a clinical care pathway chart as a patient journey in accordance with an aspect of the disclosure;

FIG. 2 shows exemplary treatment guidelines used to generate the clinical care pathway chart in accordance with an aspect of the disclosure;

FIG. 3 shows a matching of clinical markers from a patient's medical records to nodes of the clinical care pathway chart in accordance with an aspect of the disclosure;

FIGS. 4A-4C shows selection of a path from treatment guidelines in accordance with an aspect of the disclosure;

FIG. 5 shows a system for a patient care engine configured to generate a clinical care pathway chart in accordance with an aspect of the disclosure;

FIG. 6 shows a first example of a display at a graphical user interface of a display device for a patient care journey in accordance with an aspect of the disclosure;

FIG. 7 shows a second example of a display at a graphical user interface of a display device for a patient care journey in accordance with an aspect of the disclosure;

FIG. 8 shows a process for generating a clinical care pathway chart via a patient care engine in accordance with an aspect of the disclosure;

FIG. 9 shows a system for a patient care engine configured to generate patient summaries in accordance with an aspect of the disclosure;

FIG. 10 shows schematically shows a process for generating patient summaries using summarization rules in accordance with an aspect of the disclosure;

FIGS. 11A-11B show a natural language processing (NLP) pipeline for extracting clinical markers from EHRs at a clinical marker detection module of a patient care engine in accordance with an aspect of the disclosure;

FIG. 12 shows an example of a method for generating a patient journey for visual display at a display device by a patient care engine in accordance with an aspect of the disclosure; and

FIG. 13 shows an example of a method for generating patient summaries which may be included in the patient journey of FIG. 12 in accordance with an aspect of the disclosure; and

FIG. 14 shows an example of conversion of text from an EHR into a readable format in accordance with an aspect of the disclosure.

### DETAILED DESCRIPTION

The following description relates to various embodiments of patient history analysis and display of treatment guidelines based on the patient history analysis. Diagnosis and treatment of chronic diseases, such as cancer, may include many clinical markers such as procedures, check-ups, medications, results, and as well as treatment at multiple locations and clinics. A medical history of a cancer patient may store all clinical markers in a variety of file formats and, over a course of care, an amount of data corresponding to the patient's history may become large. Locating target information from the data may be time-consuming and inefficient, leading to lower quality of care and less accurate assessments, while incurring high costs.

As such, a method to organize and sort through longitudinal patient records, e.g., over an entire patient history, and efficiently indicate decisions, previous treatments, and findings directly affecting current and future selections for patient care is desired. In one example, treatment for a patient with a chronic illness may be optimized for the patient by a patient care engine configured to parse both patient medical records and a predetermined set of treatment guidelines specific to the chronic illness. The patient care engine may be a system implemented at a processor that includes a variety of software, such as modules, databases, user-interactive fields displayed at a graphical user interface of a display device, as well as algorithms determining how information is displayed at the display device. For example, the treatment guidelines may be a document providing a consensus amongst cancer care specialists regarding recommended treatments specific to diagnostic results and observations. The treatment guidelines may encompass a large amount of information that may be burdensome for a user to locate target information. By enabling the patient care engine to scan the treatment guidelines for the target information, relevant sections of the treatment guidelines may be rapidly matched to the patient's clinical markers and visually displayed, allowing the user to provide optimized care efficiently.

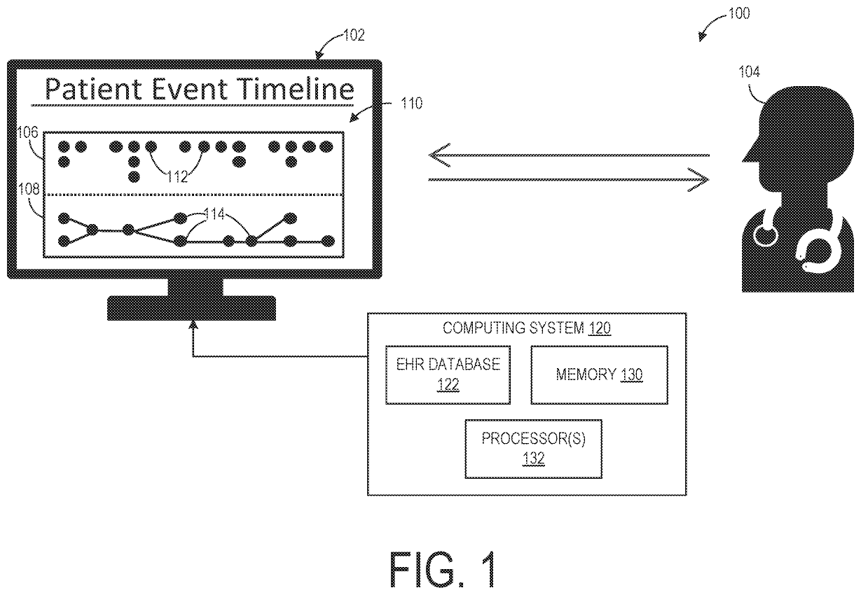

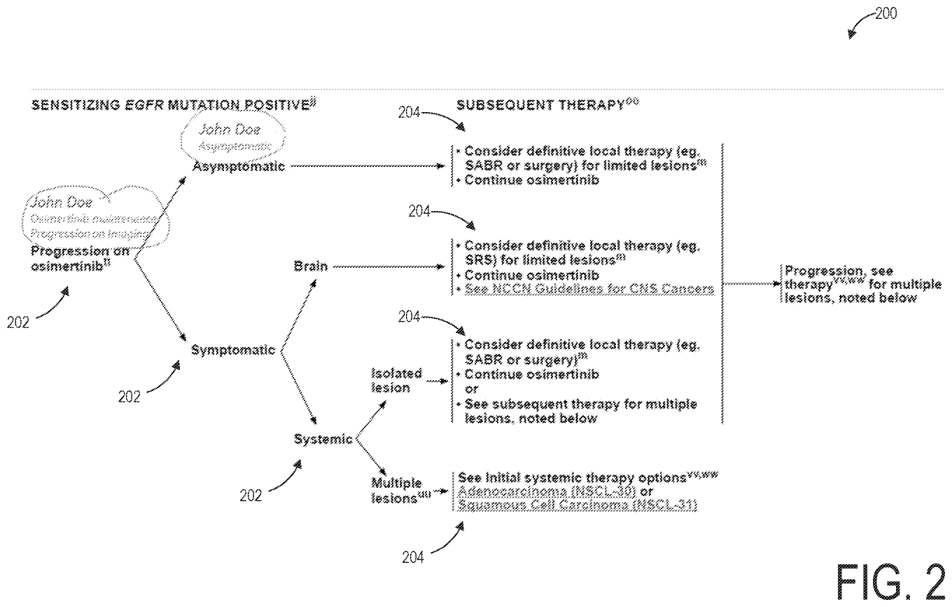

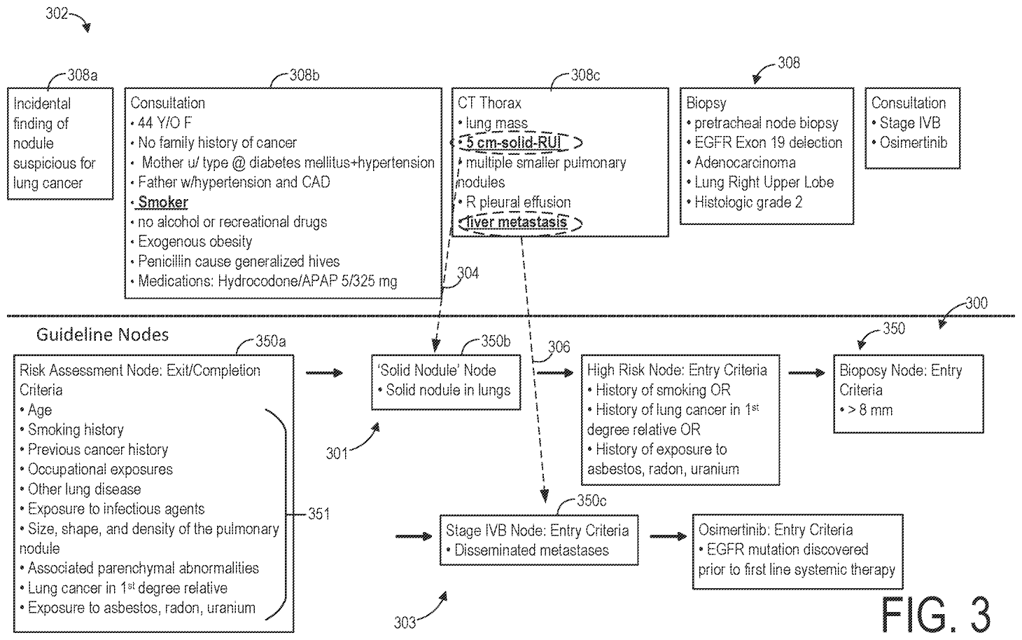

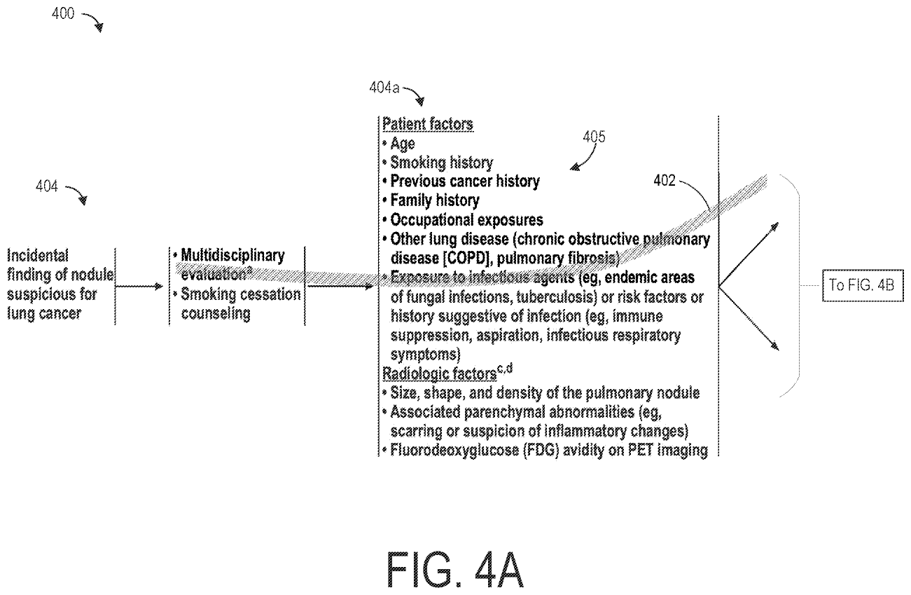

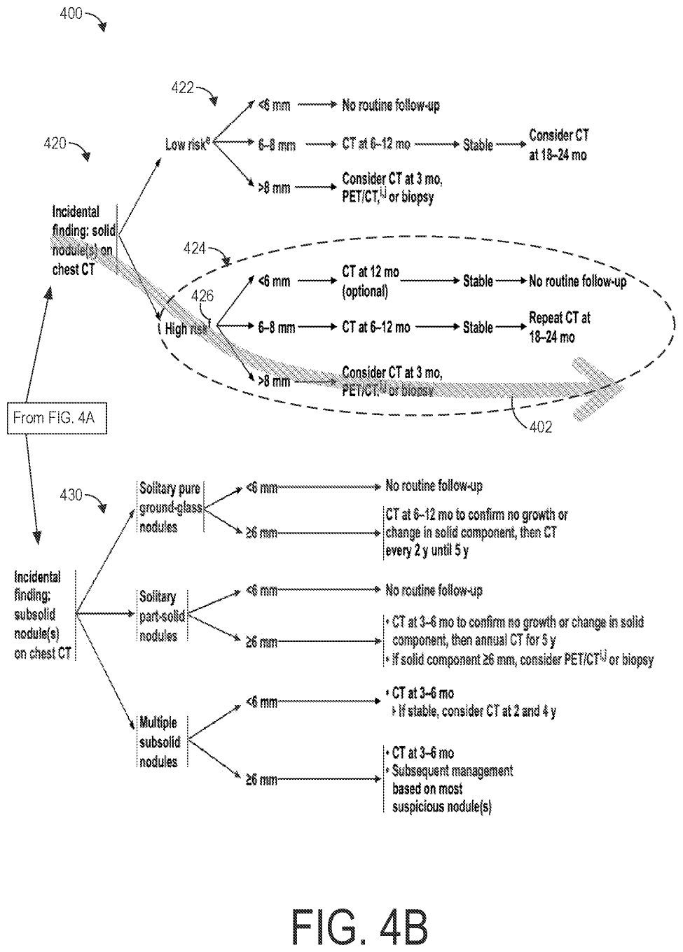

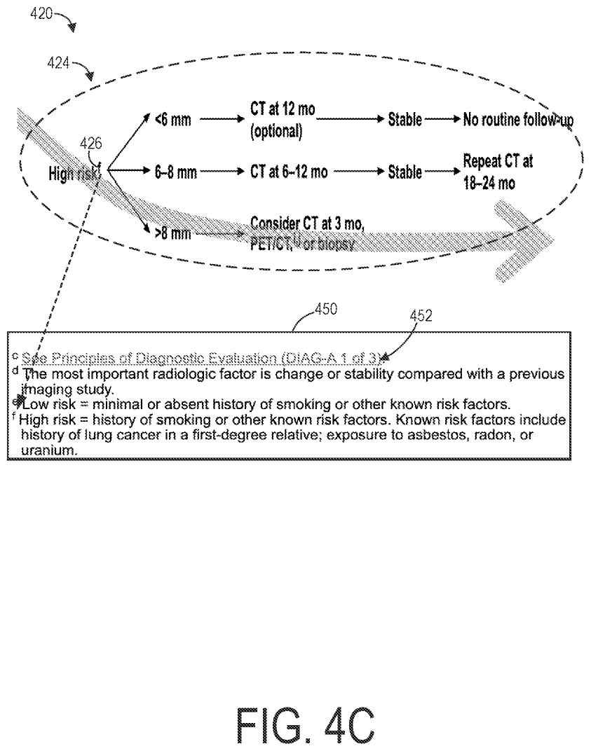

The patient medical records and corresponding treatment guidelines may be displayed in an overlaid manner, as shown in FIG. 1, to present a patient journey to the user, the patient journey showing a medical history of the patient. An example of the corresponding treatment guidelines which may be overlaid with the patient medical records is depicted in FIG. 2. As described herein and shown in FIG. 3, the corresponding treatment guidelines may be selected from the predetermined set of treatment guidelines by identifying nodes from the predetermined set of treatment guidelines and matching the nodes to clinical markers in the patient medical records. A treatment path may be determined from the matching of the nodes to the clinical markers, as illustrated in FIGS. 4A-4C.

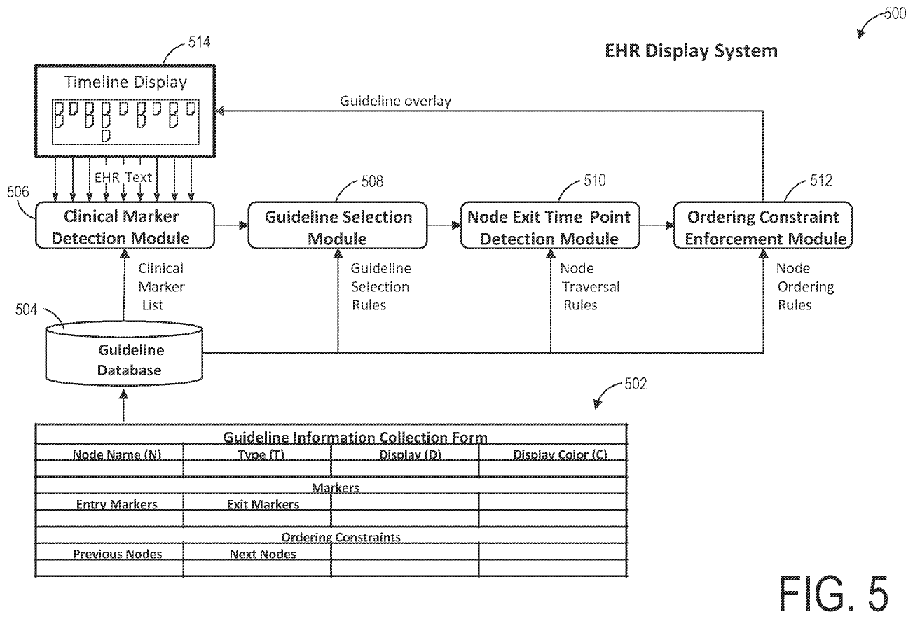

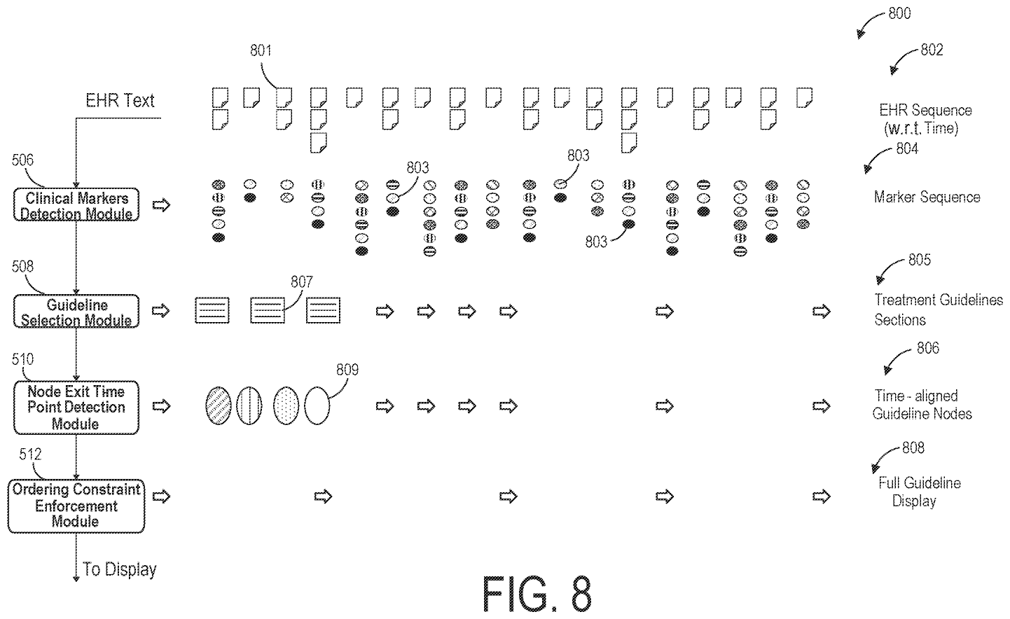

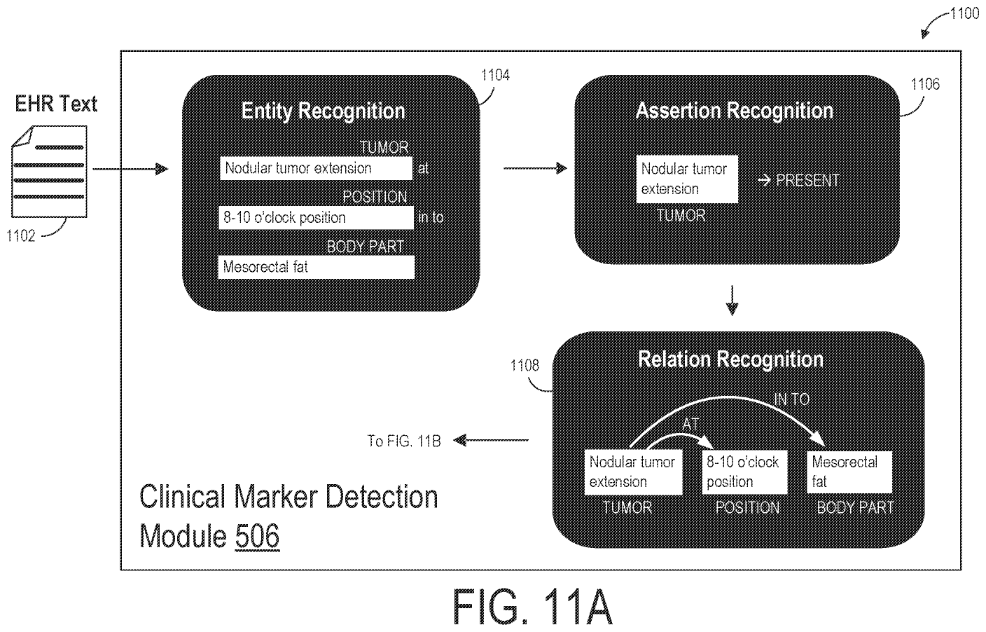

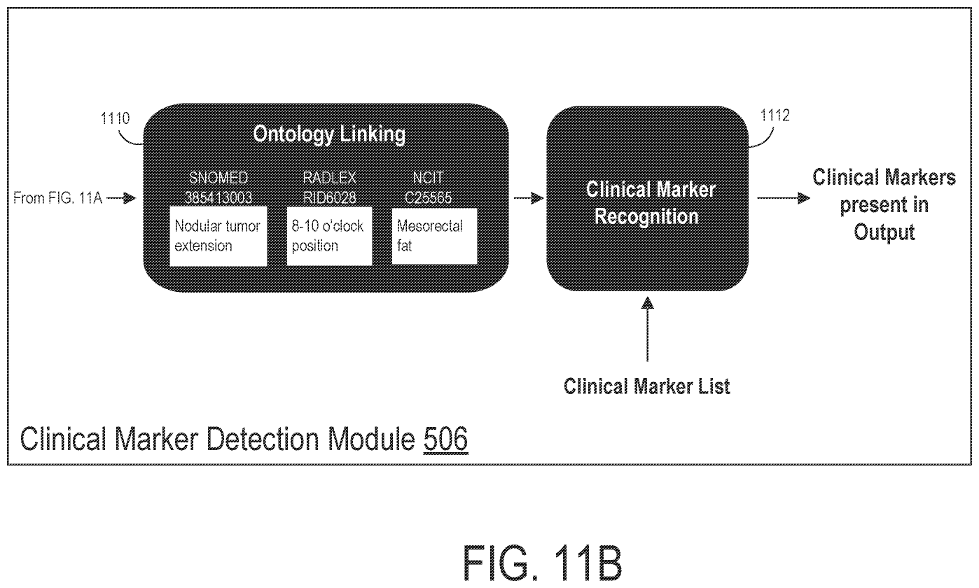

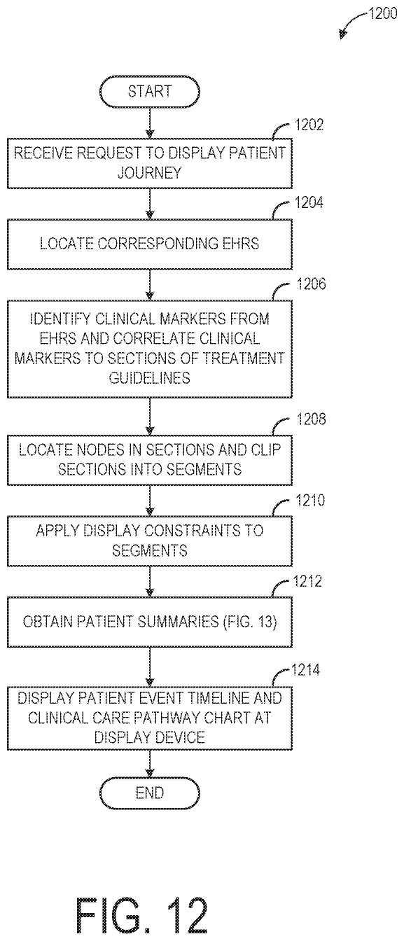

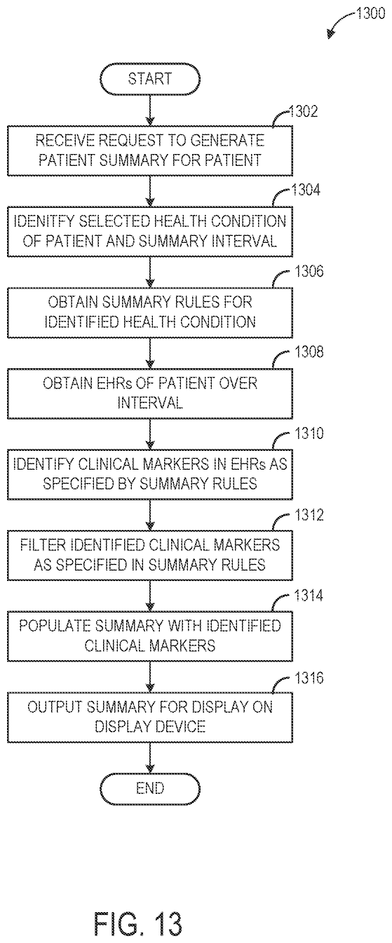

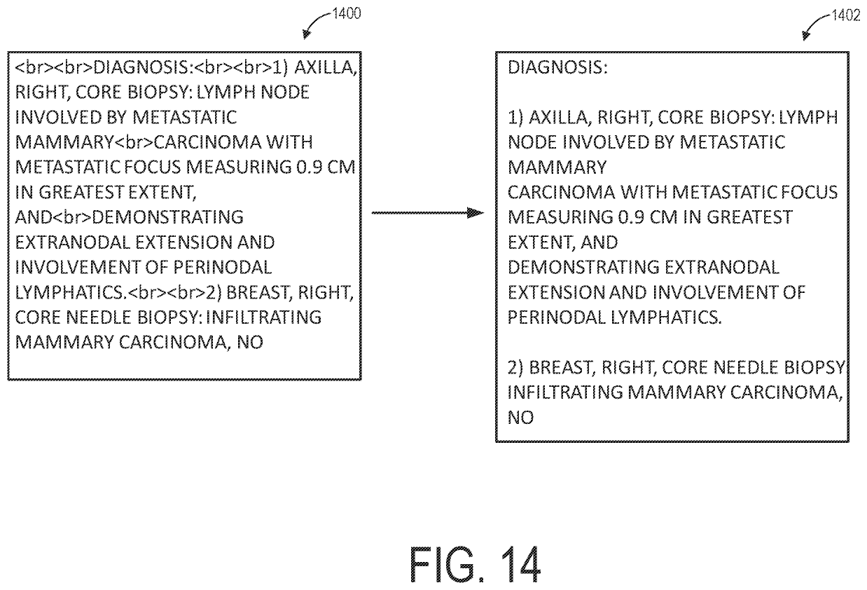

The treatment path may be provided by the patient care engine as a clinical care pathway chart. For example, the patient care engine may include a plurality of modules, as shown in FIG. 5, each module configured with algorithms for processing data according to a set of rules and a process for generating the clinical care pathway chart via the patient care engine is depicted in FIG. 8. The process may include converting text from the EHRs into a readable format, as shown in FIG. 14. Some of the modules, such as a clinical marker detection module may be configured with NLP, as shown in FIGS. 11A-11B, to extract clinical markers from EHRs in an efficient manner. The clinical markers extracted via NLP may be used to identify relevant treatment guideline segments for display in the clinical care pathway chart and to generate patient summaries for display in a patient event timeline, which may present a patient medical history indicating past and present medical events. Examples of methods for generating the patient summaries are shown in FIGS. 12-13.

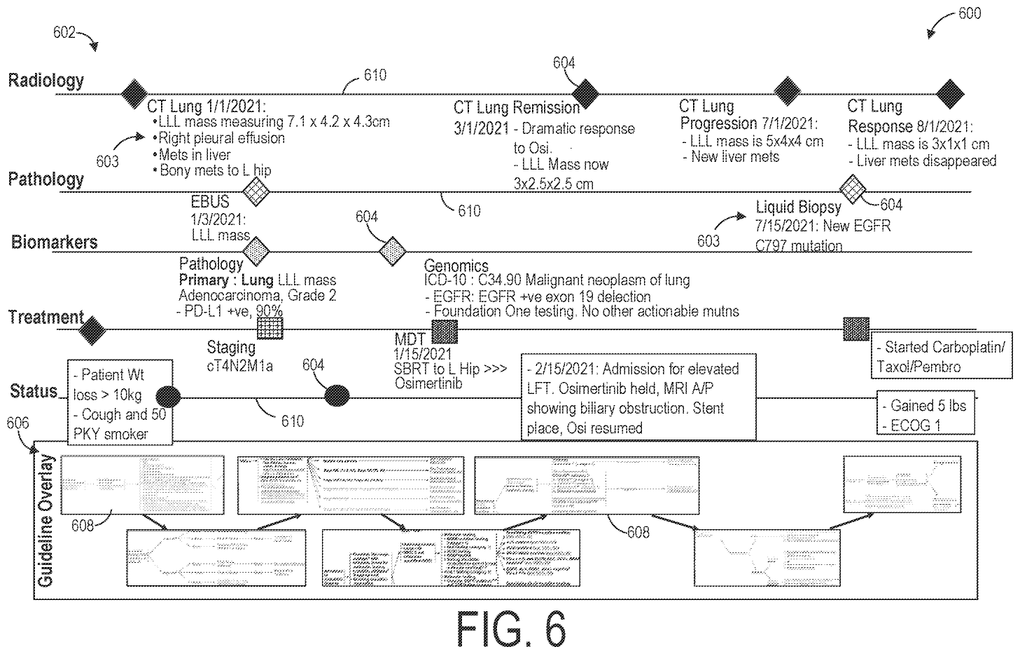

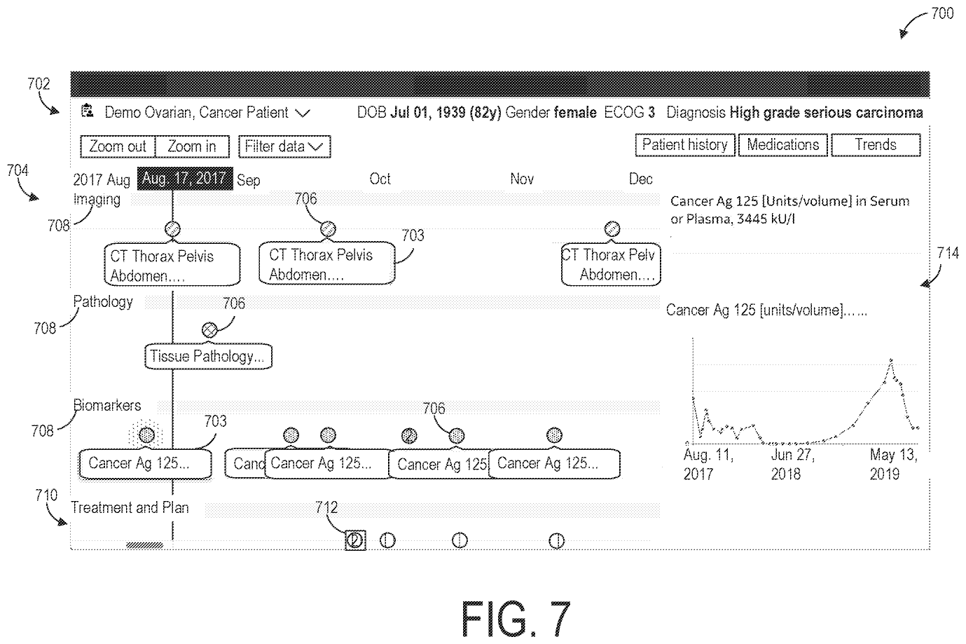

Each of the relevant treatment guidelines and the patient summaries may be listed at the patient journey and depicted as abbreviated representations in an un-launched state, where a launched state refers to interaction of the user, at a display screen displaying the patient journey, with the abbreviated representations. Upon interacting with the abbreviated representations, the abbreviated representations may be altered in appearance to display selected data corresponding to each of the abbreviated representations. The abbreviated representations may, for example, be associated with limited lists of data and depicted as graphical representations such as icons, text boxes, drop-down text boxes, images, etc. The patient journey may be displayed to the user in a comprehensive graphical representation, examples of which are illustrated in FIGS. 6 and 7.

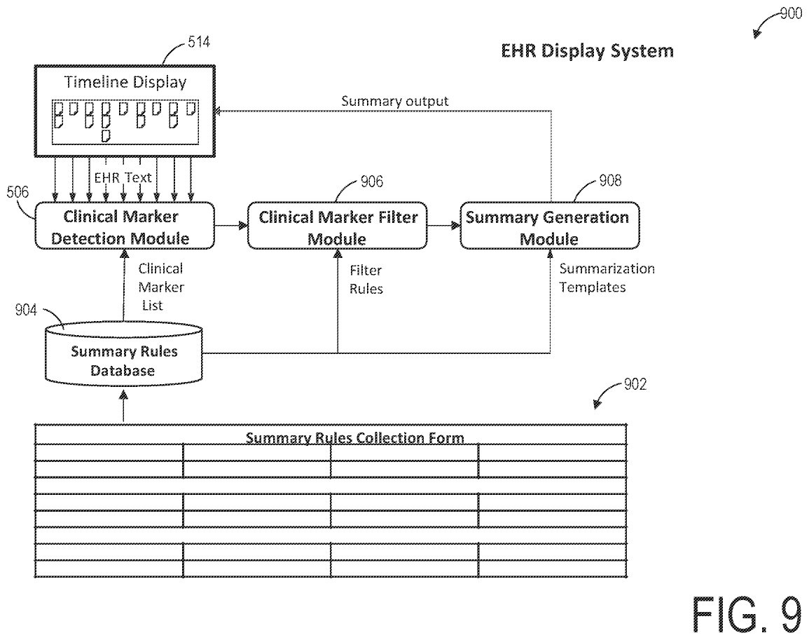

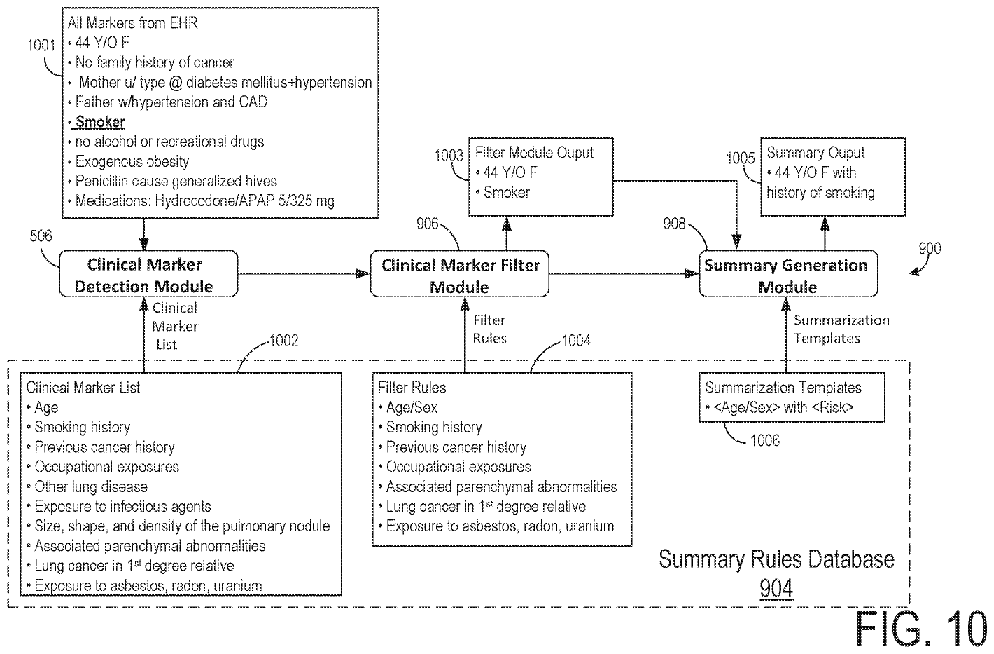

In some examples, the patient medical records may be displayed as patient summaries in the patient journey to organize and display relevant patient information in a streamlined manner. The patient summaries may be created by the patient care engine via a system shown in FIG. 9 that relies on application of summarization rules, as depicted in FIG. 10. Example of methods for generating the patient journey for display to the user is shown in FIG. 12 and an example of a method for creating the patient summaries is depicted in FIG. 13.

An example of system 100 in which a patient care engine may be implemented for displaying treatment guidelines for a patient is depicted in FIG. 1. The system 100 may include a display device 102, such as a monitor, with a display screen to present the treatment guidelines to a user 104, such as a clinician or physician. In addition, the system 100 may receive input from the user 104 via a user input device, such as a keyword, a mouse, a touch screen, etc.

The patient care engine may include instructions and algorithms for reading EHRs from the health record database and extracting information from the EHRs based on predetermined parameters and/or input rules. The information may be used to locate relevant portions of the treatment guidelines and compared to the treatment guidelines using predefined nodes, where the nodes may also be defined based on predetermined parameters and/or rules. The patient care engine may further include instructions to display the relevant portions of the treatment guidelines as the clinical care pathway chart along with a patient event timeline in a manner that allows the user 104 to readily view a current status of the patient, past treatments, and future directions of the treatment, according to the treatment guidelines.

Two types of information may be displayed at the display device 102 including clinical information 106, herein also referred to as a patient event timeline 106, overlaid with a clinical care pathway chart 108. It will be noted that the illustrated patient event timeline 106 and the clinical care pathway chart 108 are representative depictions of the information and not replicas of how the information is displayed at the display device 102. The clinical care pathway chart 108 may be overlaid with the patient event timeline 106, thereby indicating a coupling of the two types of information at relevant points and providing a rational and robust guide to patient care. Details of a visual presentation of the information is described further below.

The patient event timeline 106 may include a plurality of electronic health records (EHRs) 112 of the patient, represented as dots in a patient journey 110. The EHRs 112 may be acquired over a relatively long duration of time, such as exceeding one year, and stored in a EHR database 122 of a computing system 120 communicatively coupled to the display device 102. The patient journey 110 may be a visual presentation of a treatment path of a patient with a chronic disease, providing a visual summary of past, current, and future medical events and may include visual representations of clinical markers, EHRs, clinical findings, organized such that a user may readily find an element of interest and navigate to additional information regarding the element of interest. The visual representation may be, for example, icons, hyperlinks, images, etc. An example of a patient journey 110 is shown in FIG. 6 and described further below.

The plurality of EHRs 112 of the patient event timeline may be stored according to a time of generation and, as shown in FIG. 1, the patient event timeline 106 includes instances where multiple EHRs are generated during an event. The plurality of EHRs include various clinical markers such as diagnostic procedures, surgical procedures, therapies, administered medications, diagnostic results, etc.

As described above, data for generating the patient event timeline 106 may be acquired over time and stored in a memory 130 of the computing system 120. Generation of the patient event timeline 106 may occur during investigations of the patient and may include a variety of examination reports such as blood tests, urine tests, pathology reports, scans using various medical imaging systems such as ultrasound, magnetic resonance imaging, computed tomography systems, other radiological investigations, etc. The examination reports may be used to create the EHRs. In some examples, the patient event timeline 106 may be partitioned or segmented based on the patient's condition or treatment type, and/or the events and decisions that are clinically relevant for the user 104 may be visually indicated, e.g., by highlighting.

The clinical care pathway chart 108 may be presented according to an ordering defined by treatment guidelines. For example, the treatment guidelines may be a set of standardized protocols for treating the chronic illness developed based on consensus amongst healthcare professionals. As one example, when the chronic illness is cancer, the National Comprehensive Cancer Network guidelines may be used. Other, similar treatment guidelines may be applied to other types of chronic disease, where a primary clinical finding of a chronic disease may be tracked independently and correlated to treatment guidelines specific to the chronic disease.

The treatment guidelines may be stored at memory 130 of the computing system 120. In some examples, the patient care engine may be configured to periodically retrieve updated treatment guidelines, e.g., from a corresponding website, to be stored in addition to or, in some instances, replace the previous treatment guidelines. In other examples, the user may manually enter updated treatment guidelines for storage at memory 130.

The clinical care pathway chart 108 includes interconnected nodes 114. Relationships between the nodes 114 are indicated by connecting lines and each of the nodes may represent a junction along the treatment guidelines where a path of the clinical care pathway chart 108 may branch depending on information entered at the nodes 114. The nodes 114 may be based on diagnostic information or current or future treatment procedures include in at least one section of the treatment guidelines that is relevant to the clinical markers of the patient event timeline 106. An example of treatment guidelines 200 for cancer is illustrated in FIG. 2, showing a section of the treatment guidelines 200 where the section may be a portion of the treatment guidelines 200 relevant to a clinical finding, procedure, and/or biomarker of the patient.

The computing system 120 may include or be communicatively coupled to EHR database 122. EHR database 122 may be stored in a mass storage device (e.g., part of the computing system 120) configured to communicate with secure channels (e.g., HTTPS and TLS), and store data in encrypted form. Further, the computing system 120 is configured to control access to patient electronic medical records such that only authorized healthcare providers may edit and access the electronic health records. An EHR for a patient may include patient demographic information, family medical history, past medical history, lifestyle information, preexisting medical conditions, current medications, allergies, surgical history, past medical screenings and procedures, past hospitalizations and visits, etc.

The computing system 120 includes memory 130 and a processor(s) 132 to store and execute the methods disclosed herein to generate the patient journey 110, as well as send and receive communications, graphical user interfaces, medical data, and other information.

Memory 130 includes one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

In some examples, the computing system 120 may be implemented over a cloud or other computer network. For example, the computing system 120 is shown in FIG. 1 as constituting a single entity, but it is to be understood that computing system 120 may be distributed across multiple devices, such as across multiple servers. Further, while not shown in FIG. 1, in some examples, computing system 120 may be communicatively coupled to one or more medical data storage devices, such as a picture archiving and communication system (PACS), radiology information system (RIS), etc.

As used herein, the term “module” may include a hardware and/or software system that operates to perform one or more functions. For example, a module may include a computer processor, controller, or other logic-based device that performs operations based on instructions stored on a tangible and non-transitory computer readable storage medium, such as a computer memory. Alternatively, a module may include a hard-wired device that performs operations based on hard-wired logic of the device. Various modules shown in the attached figures may represent the hardware that operates based on software or hardwired instructions, the software that directs hardware to perform the operations, or a combination thereof.

“Modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

Turning to FIG. 2, the treatment guidelines 200 may provide standardized protocols for treating cancer and may be scanned or parsed by the patient care engine to identify suitable sections to display to the user. The treatment guidelines 200 may include various junctions 202, defining points at which more than one path is possible according to a clinical marker, as well as treatment recommendations 204. The junctions 202 and the treatment recommendations 204 may correspond to nodes of the clinical care pathway chart, e.g., the nodes 114 of FIG. 1. The nodes may have defined entry points and exit points and may be milestones along a treatment pathway of the patient, guided by the treatment guidelines. The milestones may be compared to the diagnostic and treatment information available in the patient's EHRs, e.g., the plurality of EHRs 112 of FIG. 1.

For example, an example clinical care pathway chart 300, including examples of nodes 350 are shown in FIG. 3, as well as an example of a patient event timeline 302 of a cancer patient. The clinical care pathway chart 300 and the patient event timeline 302 may be generated by a patient care engine, as described above. It will be appreciated that the clinical care pathway chart 300 shown in FIG. 3 may, in one example, be representative of how treatment guidelines may be displayed at a display device. In other examples, the clinical care pathway chart 300 may be presented in a different format. For example, each node 350 may instead be represented as an icon or displayed with abbreviated text. Similarly, the patient care timeline 302 may be displayed as shown in FIG. 3 or modified to maintain an uncluttered, organized appearance of a patient journey.

The patient event timeline 302 may include EHRs 308 arranged automatically in chronological order. For example, a first EHR 308a of the EHRs 308 may be acquired prior to a second EHR 308b of the EHRs 308 in time. The patient care engine may identify a finding of a nodule indicative of lung cancer, as indicated at the first EHR 308a, and locate matches to the finding in standardized treatment guidelines, such as the NCCN guidelines, based on defined entry and exit markers for the nodes 350. For example, an entry marker for a first node 350a of the nodes 350 may include a type of finding in the patient event timeline 302 indicative of cancer, such as lung cancer. The entry marker may trigger entry to the first node 350a which may be a risk assessment node 350a. A set of exit markers 351 is shown at the risk assessment node 350a which may be a list of target information needed to proceed to a next step, e.g., a subsequent node, in the treatment guidelines, via a first path 301 of the clinical care pathway chart 300.

The set of exit markers 351 may be used to scan the EHRs of the patient event timeline 302 to locate relevant information and provide corresponding confirmations or values to the exit markers. For example, the patient care engine may locate an age of the patient, a smoking history, a previous cancer history, etc. at the second EHR 308b. In one example, the second EHR 308b may be a patient summary created by the patient care engine according to a process for generating patient summaries described further below with reference to FIGS. 9-10. The corresponding information from the EHR may guide a subsequent route of the clinical care pathway chart of the patient, e.g., the first path 301 versus a second path 303, by directing the patient care engine to a second node 350b that is related to the first node 350a. For example, the second node 350b may be a treatment step subsequent to the first node 350a in the treatment guidelines. An entry marker to the second node 350b may include locating results of a CT scan and exit data of the second node 350b may be provided in a third EHR 308c as a finding of a 5 cm solid nodule in the patient's lungs (where an application of the finding to the clinical care pathway chart 300 is indicated by arrow 304).

The nodule in the patient's lungs may be a primary diagnostic finding and, in some examples, more than one primary diagnostic finding may be found. Additionally, in some examples, diagnostic procedures may uncover secondary diagnostic findings, such as liver metastasis, as shown at the third EHR 308c (where an application of the finding to the clinical care pathway chart 30 is indicated by arrow 306). Identification of the secondary diagnostic findings may provide entry markers which may trigger entry to a different section of the treatment guidelines specific to liver metastasis, defined as a third node 350c, as indicated by arrow 306, generating a second path 303 of the clinical care pathway chart.

In examples where more than one primary diagnostic finding is presented, each primary diagnostic finding may be tracked independently. Recommendations based on the treatment guidelines may be provided for each primary diagnostic finding, e.g., individual clinical care pathway charts may be generated for each primary diagnostic finding, which may be displayed according to user selection amongst the more than one primary diagnostic findings.

The first path 301 and the second path 303 of the clinical care pathway chart 300 may be treatment paths for different findings originating from a common node (e.g, the first node 350a). Each of the first and the second paths 301, 303 may proceed to complete subsequent nodes (e.g., provide entry markers and exit markers for each node) of each pathway until the pathways each reach points where exit markers of the nodes 350 cannot be completed. In other words, each of the paths may terminate at a node where insufficient exit markers are present to exit the node. An inability to complete the exit markers may indicate a current status of the treatment and the incomplete exit markers may represent current, ongoing procedures and/or future procedures.

In this way, relevant sections of the treatment guidelines may be located and used to assess a status of the patient's ongoing care by displaying the sections at a display device, overlaid with a patient event timeline, in real-time. In some examples, the treatment guidelines may provide vast amounts of information with many treatment paths included. Treatment guideline documents may be available in a variety of formats, such as a pdf, html, an image, etc., which may complicate efficient scanning to locate target sections, such as scanning based on a keyword or phrase. By using the patient care engine, parsing of the treatment guidelines for relevant information may be streamlined and a suitable path of care may be readily selected from many possible paths. For example, as shown in FIGS. 4A-4C, an example of a section 400 of treatment guidelines for cancer patients is depicted.

The section 400 of the treatment guidelines includes multiple junctions and outcomes. Without well-defined criteria for selecting a path at each junction, a resulting outcome may deviate from an optimal treatment path for the patient. However, by implementing the patient care engine, a specific, targeted path, as indicated by arrow 402, is selected with high accuracy, with respect to an optimal treatment path for the patient, by utilizing the patient's medical history and providing rigorous criteria for proceeding to each subsequent step.

For example, a plurality of stages 404 of treatment criteria are depicted in the section 400 of the treatment guidelines. The plurality of stages 404 are ordered sequentially and include junctions that form branches in the treatment guidelines. As an example, a patient factor stage 404a of the stages 404 may be a first junction in the section 400 of the treatment guidelines. The patient factor stage 404a may include a list of clinical markers 405, such as age, smoking history, previous cancer history, etc. The treatment guidelines may proceed to a first branch 420 or a second branch 430, as shown in FIG. 4B. Within each of the first branch 420 and the second branch 430 may be sub-branches. For example, the first branch 420 includes a first sub-branch 422 and a second sub-branch 424.

Determination of which of the first branch 420 or the second branch 430 is more suitable for the patient, and which of the respective sub-branches therein, may be achieved by parsing additional information provided in the treatment guidelines. For example, information provided in a subscript link 426 of the second sub-branch 424 of the first branch 420 may be analyzed for clinical markers corresponding to clinical markers of a patient event timeline (e.g., the patient event timeline 302 of FIG. 3) of the patient. The information corresponding to the subscript link 426 is depicted in box 450 in FIG. 4C, which describes risk factors associated with high risk of cancer. A clinical marker associated with a history of lung cancer in a first-degree relative as well as a clinical marker associated with exposure to asbestos, radon, or uranium may be matched to clinical markers provided in the patient event timeline (see the second EHR 308b of FIG. 3).

Parsing the additional information provided in the treatment guidelines may also include scanning information associated with hyperlinks embedded in the treatment guidelines. For example, a hyperlink 452 is shown in box 450, corresponding to a linked document displaying information regarding Principles of Diagnostic Evaluation. The linked document (not shown) may include relevant information for clinical markers such as sputum cytology, bronchoscopy with biopsy and TBNA, image-guided transthoracic needle core biopsy or FNA, thoracentesis, mediastinoscopy, etc. The matching of the clinical markers from the treatment guidelines, including information embedded in the treatment guidelines thus determines which information from the branches and sub-branches of the section 400 of the treatment guidelines to display in nodes of a clinical care pathway chart (e.g., the clinical care pathway chart 300 of FIG. 3) to a user.

A system 500 for a patient care engine, the system 500 configured to display a clinical care pathway chart (such as the clinical care pathway chart 300 of FIG. 3) based on patient history and treatment guidelines, is illustrated in FIG. 5. The system 500 may utilize a guideline information collection form 502 to indicate which information may be input by a user, such as a clinician or physician, for constructing the clinical care pathway chart. The system 500 further includes capabilities for obtaining information regarding a patient care journey, such as the patient's EHRs.

The guideline information collection form 502 may be provided offline, e.g., accessible to the user prior to implementation of the patient care engine, and configured as a form with user-interactive, fillable fields into which the user may either enter terms for each variable or select from a list of possible entries. For example, information requested by the guideline information collection form 502 may include a node name, a category or type of the node, confirmation of whether the node is to be displayed, and visual effects of the displayed node, such as a display color. The node name may be determined from, as one example, a table or list of possible nodes according to treatment guidelines specific to an identified chronic illness of the patient.

The guideline information collection form 502 may also include fields for defining entry and exit markers for each of the nodes. In some examples, selection of a name of the node may automatically link the name to a predetermined set of entry and exit markers from which a specific set of markers may be selected. The entry and exit markers may also be determined based on the treatment guidelines.

The guideline information collection form 502 may further include fields for defining ordering constraints (e.g., node display constraints), which may be used to modify a display of a patient care journey overlaid with a clinical care pathway chart, described further below. For example, as shown in FIG. 5, the ordering constraints may include identifying whether a named node is linked to previous or subsequent nodes according to the treatment guidelines. In some examples, the fields for the ordering constraints may be automatically filled based on the node name and/or type. For example, the node name may be automatically matched to a set of reference treatment guidelines to determine a sequential ordering of the nodes. The fields may be filled based on the ordering shown in the set of reference treatment guidelines. In other examples, the fields for the ordering constraints may be manually filled by the user. The user may refer to selected or target treatment guidelines to determine a suitable order of the nodes, according to the treatment guidelines.

In some examples, the fields of the guideline information collection form 502 may be filled manually, e.g., by the user. In other examples, the fields may be automatically filled, such as by script. For example, a set of lists may be provided, each list including information for completely filling the fields. Upon entry of a specific node name, the patient care engine may automatically identify a corresponding list and use the information from the list to fill the remaining fields.

The entries from the guideline information collection form 502 may be used in conjunction with standardized treatment guidelines for the chronic illness. The standardized treatment guidelines may be entered and stored at a guideline database 504 in a variety of formats and may include one or more sets of treatment guidelines. Entry of the treatment guidelines may be conducted offline by the user. In some examples, the treatment guidelines may be periodically updated as new findings emerge and are incorporated into the treatment guidelines. In some instances, the user may manually enter the updated treatment guidelines at the guideline database 504 when a newer version of the treatment guidelines is provided at, for example, a website or saved in a memory of a computing system at which the patient care engine is implemented. In other instances, the patient care engine may include instructions to automatically search for updated treatment guidelines and upload the updated guidelines to the guideline database 504. For example, the patient care engine may use script to recognize updated versions of the treatment guidelines based on, as one example, a new version or edition number in a title of the treatment guidelines document.

The patient care engine may retrieve data input to the guideline database 504 from the guideline information collection form 502, the patient care journey information, and the treatment guidelines and send the input data to modules such as a clinical marker detection module 506, a guideline selection module 508, a node exit time-point detection module 510, and an ordering constraint enforcement module 512. Each of the modules may utilize a set of rules defined by the guideline information collection form 502 (except for the clinical marker detection module 506) to analyze the input data and provide outputs according to the set of rules. The outputs of the modules may be used to generate the clinical care pathway chart which may be displayed at a display device 514, similar to the display device 102 of FIG. 1.

The clinical marker detection module 506 may utilize a clinical marker list, such as the list of clinical markers 405 of FIG. 4 obtained by parsing the treatment guidelines, to identify target types of clinical markers. In another example, clinical markers may be extracted from the patient's EHRs by a method such as Lookup Entity Extraction which may use a lookup table of clinical markers and textual variations of the clinical markers. As another example, a method such as Machine Learning-based Entity Extraction may rely on a trained artificial neural network to identify portions of text which corresponds to each clinical marker in one or more of the treatment guidelines and the EHRs. Various other methods of generating the clinical marker list are possible.

As described above, the clinical marker detection module 506 may be configured to locate the clinical markers from the patient's EHRs and/or the treatment guidelines to create the clinical markers list. Upon identifying one or more of the clinical markers in the patient event timeline, the clinical marker detection module 506 may search for the identified clinical markers in the treatment guidelines document to locate relevant sections of the treatment guidelines. The relevant sections may be delivered to the guideline selection module 508.

As one example, as shown in FIGS. 11A-11B, the clinical marker detection module 506 of the patient care engine may leverage machine learning algorithms via a natural language processing (NLP) pipeline 1100 for extracting clinical markers from EHRs 1102. In other examples, however, the EHRs 1102 may instead be sections of a treatment guideline, such as the section 400 of FIG. 4. The NLP pipeline 1100 may be a machine learning model implemented at the clinical marker detection module 506 and trained to perform various steps upon parsing or scanning the EHRs 1102. For example, a first step 1104 included in the NLP pipeline 1100 may include entity recognition. Entity recognition may include identifying entities from text of the EHRs 1102, such as a type of tumor, a position of the tumor, and a body part at which the tumor is located. Identified text is indicated in FIGS. 11A-11B in white boxes with black text while assignment/categorization of the identified text by the NLP pipeline 1100 is depicted in white text (below a header of each step of the NLP pipeline 1100, the header also in white text at a top of each block).

A second step 1106 of the NLP pipeline 1100 may include assertion recognition. The NLP pipeline 1100 may be trained to identify positive and negative assertions of clinical markers, such as presence or absence of symptoms, from the text of the EHRS 1102. As shown in FIG. 11A, the identified tumor may be indicated as present based on assertion recognition. It will be appreciated that the steps depicted in FIGS. 11A-11B are shown and described sequentially for clarity but are not be held to a particular chronological sequence. For example, the steps of the NLP pipeline 1100 may be executed in series or in parallel, or some of the steps may be performed concurrently while others may be performed sequentially.

The NLP pipeline 1100 may further include a third step 1108 for relation recognition, where relationships between keywords in the text of the EHRs 1102 may be identified. For example, as shown in FIG. 11A, the third step 1108 may include recognizing a relationship between the identified tumor and the body part as “in to”, and a relationship between the identified tumor and the tumor position as “at”.

Continuing to FIG. 11B, the NLP pipeline 1100 may include a fourth step 1110 for ontology linking. Concepts and categories within a domain, such as a health condition or a disease, may be recognized and paired from the text of the EHRs 1102. As such, the NLP pipeline 1100 may be trained to recognize and generate binary relationships between clinical terminology and codes. As one example, the text of the EHRs may be scanned for coded terms according to a type of medical coding and correlate a medical diagnosis code to the coded terms.

For example, as shown in FIG. 11B, a coded term may be a “nodular tumor extension”, which may be linked to a medical diagnosis code of “385413003” from SNOMED Clinical Terms (e.g., a computer-processable collection of medical terms including codes, terms, synonyms, and definitions). As another example, the coded term may be the tumor position, such as “8-10 o'clock”, which may be correlated to a RadLex code of “RID6028”, where RadLex is set of radiology terms. As yet another example, the coded term may be the location of the tumor, e.g., “mesorectal fat”, which may correspond to a NCIT code of “C25565”, where NCIT is a standard for biomedical coding and reference. By identifying the medical diagnosis codes linked to the coded term, the clinical marker detection module may parse medical information associated with the medical diagnosis codes from documents and/or databases accessible by the patient care engine. The medical information may be provided in the treatment guidelines stored in the guideline database 504 of FIG. 5.

At a fifth step 1112 of the NLP pipeline 1100, clinical markers may be recognized, e.g., clinical marker recognition, and extracted from the first, second third, and fourth steps 1104, 1106, 1108, 1110, based on the clinical marker list generated by the clinical marker detection module 506, as described above. For example, all clinical markers may be identified and extracted from the EHRs 1102 by the NLP pipeline 1100 and the clinical markers may be compared to the clinical marker list. Clinical markers that match items on the clinical marker list may be further processed by retrieving relevant treatment guidelines from the guideline database 504 and outputting the relevant sections of the treatment guidelines to the guideline selection module 508, as shown in FIG. 5.

Returning to FIG. 5, at the guideline selection module 508, a set of rules, e.g., guideline selection rules, based on the entries at the guideline information collection form 502, may be applied to the output from the clinical marker detection module 506. For example, the guideline selection rules may provide instructions for the guideline selection module to locate suitable treatment guidelines from the guideline database 504 according to the clinical markers identified by the clinical marker detection module 506. Furthermore, relevant sections of the located treatment guidelines focusing on the identified clinical markers may be extracted from the located treatment guidelines. Data regarding the relevant sections of the treatment guidelines may be sent to the node exit time-point detection module 510.

At the node exit time-point detection module 510, nodes may be identified and located in the relevant sections according to a set of node traversal rules. The node traversal rules may be generated based on the guideline information collection form 502. For example, node names and types, as entered at the guideline information collection form 502, may be used to scan the relevant sections of the treatment guidelines to locate text-based matches to the node names and types. Additionally, defined entry markers and exit markers at the guideline information collection form 502 may be incorporated to determine how much of the relevant sections corresponds to past, current, and future clinical markers of the patient's ongoing treatment plan. The relevant sections of the treatment guidelines may be clipped into segments according to the exit and entry markers that are satisfied. By evaluating whether the patient care journey includes sufficient information to satisfy the entry markers and exit markers, a path of the clinical care pathway chart may be determined according to the nodes with information that satisfies the exit markers, allowing the path to proceed to subsequent nodes until one or more treatment recommendations are attained.

The satisfied nodes and treatment recommendations may be sent to the ordering constraint enforcement module 512 to be modified for display at a display device according to the entries at the guideline information collection form 502, such as which nodes are to be displayed, the color, and the ordering constraints, e.g., which nodes are previous and sequential to a specific node. For example, a sequence of the nodes may be adjusted according to the ordering constraints such that the order adheres to an order shown in the treatment guidelines. As an example, a biopsy node is depicted as a last node in the first path 301 of the clinical care pathway chart of FIG. 3, positioned after the second node 350b which corresponds to a finding of a solid nodule. However, the biopsy may have been performed prior to a CT scan confirming the solid nodule in the lungs in the patient's EHRs. Thus the order of the nodes may be independent of the chronology of the clinical markers in the patient's EHRs, in some examples.

The segments of the treatment guideline may be displayed as the clinical care pathway chart at the display device 514 according to the guideline information collection form 502 and information processed by the modules. At the display device 514, the clinical care pathway chart may be overlaid with the patient event timeline. In some examples, each of the clinical care pathway chart and the patient event timeline may be depicted as graphical representations, such as icons, drop-down text boxes, images, etc. For example, the clinical care pathway chart, depicted as a series of nodes as shown in FIG. 3 with exit markers indicated, may be shown alongside the patient event timeline such that the nodes are matched with corresponding clinical markers. The clinical markers may be ordered according to a sequence of the nodes, where the sequence of the nodes is determined from the treatment guidelines. In another example, the relevant section of the treatment guideline may be digitized and EHRs corresponding to nodes of the relevant section shown may be overlaid onto the representation of the relevant section of the treatment guideline displayed. Input values for the nodes may also be shown as a decision box to allow the user to make an informed selection amongst recommended treatments.

A first example of a visual display of a patient journey 600 is shown in FIG. 6. The patient journey 600 may be presented at a display device and may include a patient event timeline 602 depicting a plurality of clinical markers 604 extracted from EHRs of the patient. The plurality of clinical markers 604 may be annotated with text, e.g., summaries 603, providing information regarding a corresponding clinical marker. In some example, the summaries 603 may be abbreviated representations of data, such as icons, images, drop-down text boxes, etc., from the patient event timeline 602, with each of the summaries listing an amount of the data selected based on a correlation to the plurality of clinical markers 604. As such the abbreviated representations may be presented in an unlaunched state at the patient journey 600 and, when engaged and/or interacted with by a user, may be launched to display the selected data, as shown in the summaries 603. The patient event timeline 602 may be ordered according to a clinical care pathway chart 606, shown below the patient event timeline 602.

The patient event timeline 602 may include segments 608 of treatment guidelines, the segments 608 selected based on matching of the treatment guidelines to nodes identified by a patient care engine, as described above. The segments 608 are connected by arrows to denote a sequential ordering of the segments 608. It will be appreciated that the segments 608 are depicted arranged in a non-linear layout in FIG. 6 for illustrative purposes and may, in practice, be arranged aligned along a horizontal axis to correspond with specific clinical markers of the plurality of clinical markers 604 along a vertical axis. In other words, the patient journey 600 shown in FIG. 6 is not ordered chronologically along the horizontal axis and may be displayed according to a different layout than depicted.

The plurality of clinical markers 604 are shown grouped according to a type of clinical marker. Each group of clinical markers may be displayed as graphical representations along a lane 610 and spaced along the lane 610 to align with a corresponding segment of the segments 608 of the clinical care pathway chart 606. As such, the patient event timeline 602 and the clinical care pathway chart 606 are displayed in a clear and organized layout with minimal amounts of text that may otherwise lead to a cluttered appearance of the patient journey 600. The user may easily locate clinical markers or segments of the treatment guidelines and also identify a relationship between the clinical markers and segments.

In some examples, the patient care journey may include clinical markers that deviate from the treatment guidelines. When such instances are identified, a visual indicator, such as a message, and/or a visual modification to the EHR, such as highlighting or a color change, may be used to notify the user of the deviation.

In other examples, the clinical care pathway chart 606 may also be depicted as abbreviated representations, as described above for the patient event timeline 602, presented to the user as visual objects, such as icons, text boxes, images, etc. The abbreviated representations of the clinical care pathway chart 606 may be displayed proximate to a corresponding clinical marker of the clinical markers 604 when in an unlaunched state. Alternatively, the clinical care pathway chart 606 (and/or the patient event timeline 602) may be visible in the unlaunched state until the user interacts with the clinical markers 604. For example, when the user adjusts a position of a cursor over one of clinical markers 604, corresponding abbreviated representations of the patient event timeline 602 and/or the clinical care pathway chart 606 may appear. If the user selects one of the abbreviated representations, the abbreviated representation may be launched to display the selected data associated therewith, which may be one of summaries 603 and/or one of the segments 608 of the treatment guidelines. As such, the patient journey 600 displayed at the display screen provides the user to directly reach, e.g., acquire to observe, data listed in each of the patient event timeline 602 and the clinical care pathway chart 606 in real-time and with reduced processing of data.

A second example of a visual display of a patient journey 700 is shown in FIG. 7. The patient journey 700 may be presented at a display device and may include a header section 702 at a top of the visual display that relays patient information, such as type of patient, date of birth, gender, etc. The visual display of the patient journey 700 may also include a patient event timeline 704 displayed below the header section 702, similar to the patient event timeline 602 of FIG. 6, the patient event timeline 704 depicting a plurality of clinical markers 706 extracted from EHRs of the patient and arranged along lanes 708 corresponding to a lane indicating a type of corresponding clinical markers of the plurality of clinical markers 706. The plurality of clinical markers 706 may be annotated with text, e.g., summaries 703 listing patient-specific medical events and notes, describing a corresponding clinical marker. As described above with reference to FIG. 6, the summaries may be displayed as abbreviated representations of selected data associated with the clinical marker when in an unlaunched state. For example, as shown in FIG. 7, only a portion of the selected data may be visible in the summaries 703 when the summaries are unlaunched. Upon engagement by a user, the summaries 703 may be launched and expanded to display an entire list of data associated with each of the summaries 703.

A clinical care pathway chart 710 may be presented below the patient event timeline 704 (only partially shown in FIG. 7). In one example, the clinical care pathway chart 710 may include abbreviated representations, such as user-interactive icons 712 which represent segments of treatment guidelines selected according to the plurality of clinical markers 706. The user-interactive icons 712 may be annotated with text summarizing a corresponding segment of the treatment guidelines. Alternatively, text annotations of each of the user-interactive icons may remain hidden from view until a user interacts with one of the user-interactive icons, upon which a text pop-up may be displayed.

In the patient journey 700, the plurality of clinical markers 706 may be ordered chronologically along the patient event timeline 704. In some examples, as shown in FIG. 7, the user may select a time point along the patient event timeline 704, which may result in display of a treatment graph 714 to a right of the patient event timeline 704. The treatment graph 714 may show a record of treatment of the patient from the selected time point forwards, according to treatments recommended based on the clinical care pathway chart 710. Display of the treatment graph 714 may vary according to the selected time point.

It will be appreciated that the visual displays of the patient journeys shown in FIGS. 6 and 7 are non-limiting examples of how the patient event timeline and clinical care pathway charts may be presented to a user. Variations in how the visual displays are configured may deviate from those shown without departing from the scope of the present disclosure.

A process 800 for processing information from a patient's EHRs 801 via a patient care engine is depicted schematically in FIG. 8. Various modules are shown along a left hand side of FIG. 8 with arrows indicating progression of the process 800. Results output from and corresponding to each of the modules are indicated along a right hand side of FIG. 8. At 802 of the process 800, the EHRs 801 may be converted to a readable format to allow clinical markers 803 to be extracted. For example, as shown in FIG. 14, a section 1400 of an EHR may include markers such as “<br>” as coded instructions for transforming the section 1400 into a converted section of text 1402. In the converted section of text 1402, the markers indicate where line breaks are to be added, thereby organizing an appearance of the converted section of text 1402 to be more organized and comprehensive. In other examples, text of the EHRs may be parsed or crawled to identify keywords corresponding to the clinical markers 803. The EHRs 801 may be stored in a time-ordered sequence (e.g., chronologic based on collection time). The process 800 further includes processing of the EHRs 801 using one or more of the modules shown in FIG. 5, including the clinical marker detection module 506, the guideline selection module 508, the node exit time-point detection module 510, and the ordering constraint enforcement module 512. For example, a marker sequence may be generated at 804 by the clinical marker detection module 506 which may include differently colored clinical markers 803, (indicated by different shading in FIG. 8) each color (or shading) representing a different type of clinical marker.

At 805 of the process 800, the marker sequence may be processed according to guideline selection rules at the guideline selection module 508. The guideline selection rules may be input by a user, e.g., via the guideline information collection form 502 of FIG. 5, and used to identify corresponding sections, e.g., sections of text 807, from treatment guidelines stored at a database accessible by the patient care engine, such as the guideline database 504 of FIG. 5. The sections may be forwarded to the node exit time-point detection module 510.

At 806, the process 800 includes comparing the marker sequence to nodes 809 from the sections of the treatment guidelines at the node exit time-point detection module 510. The nodes 809 may be presented with a visual attribute to indicate a state of each node. For example, each node may include data satisfying exit markers, e.g., the node is a past node, or the node may be a currently entered node or a future node. As an example, the nodes 809 may be shown in different colors (e.g., represented by different shading in FIG. 8) or shapes, according to the state of the node. In addition, the nodes 809 may be ordered according to the treatment guidelines. At 808, the process 800 includes displaying a full guideline generated by the ordering constraint enforcement module 512. For example, the full guideline may be ordered according to the treatment guidelines and displayed at the display device.

In this way, information from a patient's medical records may be extracted and matched with treatment guidelines for a chronic illness to provide treatment recommendations efficiently. By implementing a patient care engine configured to identify clinical markers from the medical records and rapidly locate relevant sections of the treatment guidelines based on the clinical markers, an amount of time spent sorting through large quantities of information may be reduced while an accuracy of the treatments may be increased.

In order to generate a patient event timeline and clinical care pathway chart, as shown in FIGS. 3, 6, and 7, to be displayed at a display device, efficient identification of relevant data from a large quantity of patient EHRs may be demanded. A process for organizing and locating data from the patient EHRs may be enabled by generating and displaying a periodic summary of the patient. Generation and display of the periodic summary may allow large volumes of data to be scanned and relevant information to be extracted from the data according to pre-set time intervals. The summaries may therefore be maintained current and relevant, and enable healthcare providers to readily observe trends in guideline-relevant parameters (such as tumors, demographics, treatment, response, comorbidity, etc.). A cognitive burden on a care provider team for organizing and locating relevant information from the patient EHRs may therefore be reduced and the patient event timeline and clinical care pathway chart may be generated in a rapid, streamlined manner.

A patient care engine configured to generate the patient event timeline and clinical care pathway chart may also be configured to create the summaries from a common EHR database, such as EHR database 122 of FIG. 1. An example of a system 900 for a patient care engine is illustrated in FIG. 9, the system 900 configured to analyze patient history and generate summaries based on the patient history (as shown at the second EHR 308b of FIG. 3).

The system 900 shares components in common with the system 500 of FIG. 5, such as the clinical marker detection module 506 and the display device 514, which will not be re-introduced for brevity. A portion of the system 900 is also depicted in FIG. 10, along with information input and output from modules of the system 900. Included in system 900 is a summary rules collection form 902, which may be displayed on the display device 514, as an example, and may be configured to receive user input. A user (e.g., a clinician) may input details of summarization rules into fillable fields of the summary rules collection form 902.

In one example, the user may input a summarization rule to summarize any medical event, lab result, patient status/demographic information, medicines or treatments administered, pathology results, imaging results, etc. The user may enter a plurality of summarization rules for any preference of specificity relating to the patient and EHRs of the patient. The user may input summarization rules to summarize data from the EHRs based on a medical event addition, a medical event absence, a medical event reordering, a medical event parameter change, and the like. In one example, the summarization rules may be partially auto-generated, such that if the user is searching for clinical markers relating to lung cancer, all clinical markers related to lung cancer according to known medical guidelines may be automatically included in the summarization rules. The summarization rules may include three components, as shown in FIG. 10: a clinical marker list 1002, filtering rules 1004, and summarization templates 1006, which may each be stored at a summary rules database 904 depicted in FIGS. 9 and 10.

The clinical marker list 1002 may be generated, may include various markers, and may be used in the system 900 as described above, with reference to FIG. 5. For example, portions of text may be identified from the EHRS, as indicated at box 1001 of FIG. 10, which corresponds to clinical markers extracted from the EHRs. The filtering rules 1004 may be a set of predefined parameters, e.g., as input by a user or determined automatically from the guidelines, to filter an output received at a clinical marker filter module 906 from the clinical marker detection module 506. The set of predefined parameters may include, for example, an age/sex of the patient, smoking history, previous cancer history, etc. The filtering rules 1004 may be used by the clinical marker filter module 906 to generate a filter module output 1003, as shown in FIG. 10 and described further below.

The summarization templates 1006 may be predefined templates for generating a summary output 1005, as indicated by a box, at a summary generation module 908. For example, the summarization templates 1006 may be textual phrases with empty slots to be filled with values sent from the clinical marker filter module 906 to complete the summary output. Thus, the empty slots are filled based on the clinical markers that are not removed by the clinical marker filter module 906. As one example, as shown in FIG. 10, values for age/sex and risk of the patient may be designated variables for which values may be entered by the summary generation module 908 based on the filter module output 1003 received from the clinical marker filter module 906.

In some instances, the generation of the summarization rules may include auto-generation of the summarization rules from treatment guidelines, such as the section 400 of the treatment guidelines shown in FIG. 4, rather than direct input from the user. For example, text of the section 400 may be parsed or crawled, for example, to locate the patient factor stage 404a of the section 400 relevant to treatment of a specific chronic illness, such as lung cancer. The user may further provide additional search terms, such as “risk assessment”. The patient factor stage 404a of the section 400 may be parsed or crawled to extract clinical markers described in the patient factor stage 404a.

The filtering rules 1004 may be auto-generated based on one or more decision factors following a section of treatment guidelines, such as the section 400 of the treatment guidelines of FIG. 4A, used to generate the clinical marker list 1002. For example, the first branch 420 of the section 400 of the treatment guidelines of FIG. 4B may be evaluated (e.g., parsed or crawled) to determine the filtering rules 1004. As an example, the determination of whether a patient is high risk may be based on family history of lung cancer, toxin exposure, and history of smoking. Thus, the clinical marker list 1002 may be a more exhaustive list of clinical markers that may be filtered to obtain target clinical markers based on how those clinical markers are used downstream in the guidelines. By parsing or crawling the guidelines, the clinical marker list 1002 and the filtering rules 1004 may be auto-generated.

Returning to FIG. 9, the summary rules database 904 may receive a query based on the input from the summary rules collection form 902. In one example, the summary rules collection form 902 may translate human language into a query so that the summary rules database 904 may be searched. The summary rules database 904 may output clinical markers to be extracted and/or rules for filtering extracted clinical markers according to the rules defined via the summary rules collection form 902 when a summary is to be generated. The summary rules database 904 may communicate with the clinical marker detection module 506 by sending the clinical marker list 1002 as search criteria in the EHRs of the patient. The summary rules database 904 may also communicate with the clinical marker filter module 906 by sending the filtering rules 1004 to indicate information requested by the user to be included or excluded in the summaries. Additionally, the summary rules database 904 may communicate with the summary generation module 908 by sending the summarization templates 1006 to create human language summaries out of any information to be displayed to the user.

The clinical marker filter module 906 may search through the EHRs and/or extracted clinical markers sent by the clinical marker detection module 506 to filter out any information unrelated to clinical markers specified by the filtering rules 1004 stored in the summary rules database 904. Clinical markers that are not related to a diagnosis or treatment of the current patient, or are otherwise determined not to be relevant to the current summary, may be removed. In one example, the patient may be classified as a high risk patient for lung cancer and associated clinical markers for the high risk determination, according to selected guidelines, may include age, smoking status, family history of cancer, and exposure to toxins. The clinical marker detection module 506 may extract clinical markers in an EHR from a patient visit where the patient age, smoking status, family history of cancer, and exposure to toxins were recorded. If the current patient has no family history of cancer, the clinical marker filter module 906 may filter the clinical markers with the indication that the patient has no family history of cancer, such that only the clinical marker(s) relevant for the high risk determination (e.g., age and smoking status) are included in the corresponding summary output 1005.

The clinical marker filter module 906 may communicate with the summary generation module 908 by sending clinical markers and any associated information from the EHRs that pass the filter rules 1004 sent by the summary rules database 904, as requested by the user. The clinical marker filter module 906 may expedite generation of the summary output 10005 by removing EHRs and information from the included EHRs that are not relevant to the current diagnosis, treatment, and guidelines, thereby presenting a concise and comprehensive summary to the user when displayed at the display device 514.

The summary generation module 908 may take the clinical markers filtered by clinical marker filter module 906, e.g., via the filter module output 1003, and the summarization templates 1006 from the summary rules database 904 to create summaries of the EHRs to be presented to the user. In one example, the summarization templates 1006 may include natural language sentences with regions configured to receive the filtered clinical markers from the summary generation module 908. In another example, the summary generation module 908 may receive two inputs: the filter module output 1003 from the clinical marker filter module 906 and the summarization templates 1006.

The summary generation module 908 may send the summary output 1005 to the display device 514, where the summary output 1005 may be presented similar to the second EHR 308b of FIG. 3. In one example, the summary output 1005 may be displayed as part of or along with a patient event timeline, as shown in FIG. 3. The summary output 1005 may include one or more summaries created by the summary generation module 908 and/or a visual representation of the one or more summaries (e.g., an icon, a text pop-up, etc.), such that a given summary may be viewed when the corresponding visual representation is selected. The summaries, when viewed, may include links to the actual EHRs from which the clinical markers were extracted, at least in some examples, or the user may select a displayed EHR from the timeline for more information if the user requests more information. In one example, summaries for each interval of the timeline of the current patient may be displayed at a location corresponding to that interval, whether above, below, or on the timeline.

In this way, a current health condition of a patient may be determined efficiently and accurately by configuring a patient care engine to generate summaries highlighting notable events and statuses of the patient and presenting the summaries to a user in a concise and comprehensive visual display. The summaries may be exhibited in a patient event timeline that is overlaid with a clinical care pathway chart to allow the user to easily obtain information regarding past and current treatments prescribed to the patient and corresponding effects of the treatment. Large quantities of data which would otherwise demand burdensome durations of time and cognitive energy for the user to examine, analyze, and compile, leading to increased likelihood of errors, may be rapidly processed and updated. Relevant information is identified and extracted for display, thereby mitigating obscuring of more useful data by less useful data. A user may thereby assess an adherence of the patient's treatment to standardized, pre-determined treatment guidelines.

The technical effect of generating a patient medical path, e.g., a patient journey, to display patient history and treatment via a patient care engine, as described herein, is that relevant information may be automatically extracted from a large pool of information and sorted to be presented to a user more quickly and in real-time in a comprehensive and concise visual representation displayed to the user with less processing. In response to a request for patient information, a timeline of health events may be automatically generated and correlated to a treatment pathway in real-time, based on common clinical markers, thus avoiding additional processor processing of data that is not relevant to the inquiry. The timeline may be overlaid with the treatment pathway in the visual representation, allowing the user to readily observe a current and past condition of the patient, previous treatments, current treatments, and recommended treatments. The information may be retrieved by parsing and crawling EHRs and treatment guidelines stored in one or more database to rapidly output the patient journey. Creation of the patient journey, in some examples, may be aided by leveraging machine learning algorithms, such as NLP, to generate summaries and select treatment guideline segments displayed in the timeline. As a result, clinical workflow is faster and more efficient, allowing the user to spend less time searching for and retrieving patient information and more time directly interfacing with the patient and a clinical care team.

Furthermore, a display of the patient journey at a display screen of a display device may gather a pool of information offered by the timeline and the treatment pathway and present the information as abbreviated representations. When the abbreviated representations are in an unlaunched state, selected data listed by each of the abbreviated representations may be at least partially hidden from view, thereby condensing a visual footprint of the abbreviated representations. When engaged with by the user, e.g., the user clicks on or position a cursor over thereat, one or more of the abbreviated representations may be launched to expand and display the corresponding list of selected data. The selected data may be a limited quantity of data determined to have a high correlation with a clinical marker of interest. As a result, the display screen may maintain an organized, comprehensive appearance, allowing the user to quickly retrieve desired information. The display of the patient journey enables associations between lists of selected data to be identified in real-time and presented at the display in a manner that allows the user to directly access the information without spending prolonged periods of time locating the information.

An example of a method 1200 is shown in FIG. 12 for generating a patient journey for visual display at a display device by a patient care engine. The method 1200 may include displaying patient summaries, which may be provided as shown in FIG. 13 by a method 1300 for generating patient history summaries. Methods 1200 and 1300 may be carried out according to instructions stored in memory of a computing device, such as the computing system 120 of FIG. 1, which may be executed by a processor (e.g., processor 132 of FIG. 1) of the computing device via a patient care engine.

Turning first to FIG. 12, at 1202, method 1200 includes receiving a request to display a patient journey of a patient at a graphical user interface of a display device. The request may be indicated by submission of entries at a guideline information collection form, such as the guideline information collection form 502 of FIG. 5, as an example. Upon receiving the request, at 1204, EHRs for the patient are located by the patient care engine from a database storing patient records, such as EHR database 122 of FIG. 1. In some examples, the located EHRs may be converted to a format recognized by the patient care engine. In other examples, text of the EHRs may be crawled or parsed to identify keywords and/or characters.

At 1206, method 1200 includes identifying clinical markers from the EHRs and correlating the identified clinical markers to sections of one or more treatment guidelines. Identifying the clinical markers may include generating a clinical markers list from the EHRs, as described above, at a clinical marker detection module such as the clinical marker detection module 506 of FIGS. 5 and 8-10. Corresponding clinical markers may be located from treatment guidelines stored at a database of the patient care engine, such as the guideline database 504 of FIG. 5, by a guideline selection module (e.g., the guideline selection module 508 of FIGS. 5 and 8) of the patient care engine. Sections of the treatment guidelines providing information associated with the clinical markers may be extracted.

Method 1200 includes, at 1208, locating nodes in the sections of the treatment guidelines and clipping the sections into segments. For example, criteria such as node names, types, display parameters, as well as entry and exit markers, input by the user into the guideline information collection form, may be used to identify nodes in the sections of the treatment guidelines via a node exit time point detection module (as shown in FIGS. 5 and 8). Segments corresponding to the nodes and satisfying the input criteria, may be selected from the sections and the sections may be clipped to selectively show the segments.

At 1210, method 1200 includes applying display constraints to the segments and nodes of the treatment guidelines. For example, the segments corresponding to nodes to be displayed (as defined by the entries to the guideline information collection form) may be ordered for display according to a sequence shown in the treatment guidelines and to input criteria for displaying the segments, including colors, shapes, etc., of visual representations of the nodes. The display constraints may be applied to the segments and nodes by an ordering constraint enforcement module, for example, as shown in FIGS. 5 and 8.

Method 1200 further includes obtaining patient summaries at 1212. For example, the patient summaries may be generated according to method 1300 of FIG. 13 and patient summaries corresponding to the segments and nodes, e.g., based on time, diagnosis, prescribed treatment, etc., may be selected by the patient care engine.

At 1214, method 1200 includes constructing a visual display of the patient care journey, the visual display including a patient event timeline incorporating the selected patient summaries and a clinical care pathway chart incorporating the segments and nodes, and displaying the patient care journey at a display device. For example, the patient event timeline and the clinical care pathway chart may be overlaid at a display screen of the display device, allowing relationships between patient summaries, segments, and nodes to be readily observed. Method 1200 then ends.

Turning now to FIG. 13, at 1302, method 1300 includes receiving a request to generate patient summaries for the patient. The request may be received via user input (e.g., a clinician may select a user interface button displayed on a display device to request a patient history summary for a selected patient be displayed, where the user interface button may be displayed with a timeline for that patient) or automatically in response to a timeline for a patient being displayed/launched. At 1304, a selected health condition of the patient is identified and a summary interval is determined. The health condition of the patient may be identified based on user input, via analysis of patient EHRs, or based on preconfigured settings. The summary interval may dictate which information (temporally) is to be included in the summary, e.g., the past month, 3 months, year, since a previous diagnosis or treatment, etc. The summary interval may be determined based on user input or preconfigured settings.

At 1306, summary rules are obtained for the identified health condition. The summary rules may be stored in a summary rules database, such as the summary rules database 904 of FIG. 9, and the summary rules may be output from the database according to the identified health condition. In this way, different summary rules may be output for different patient conditions, e.g., a first set of rules for lung cancer, a second set of rules for breast cancer, etc. At 1308, the EHRs for the patient over the interval are obtained. The EHRs may be obtained from an EHR database, e.g., EHR database 122 of FIG. 1.

At 1310, clinical markers in the EHRs are identified as specified by the summary rules. For example, the summary rules may include a set of clinical markers that, if present in an EHR, are to be identified and extracted. The clinical markers may include certain test results, measurements, pathology findings, patient information, treatments given, tumor staging, presence of secondary tumors, and the like. The text in the EHRs may be processed to identify and extract the clinical markers. For example, as described above, methods to extract the clinical markers include using various extraction methods that rely on lookup tables, neural networks, etc., to identify the clinical markers in the text of the EHRs. At 1312, the identified clinical markers (as extracted from the EHRs) are filtered as specified by the summary rules. As explained previously, the extracted clinical markers may be filtered to remove clinical markers that are not relevant to the patient condition or guidelines for treating the patient condition.

At 1314, a patient history summary is populated with the identified, filtered clinical markers. A summary template may be populated with the clinical markers, which may format the clinical markers into natural language or another suitable format. At 1316, the patient history summary is output for display on a display device. The summary may be displayed along with a timeline of patient events and/or EHRs, at least in some examples. Method 1300 then ends.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for a computing system, comprising: a display screen configured to display a patient medical path listing one or more of treatment guidelines and patient medical history, and additionally being configured to display abbreviated representations of at least one of the treatment guidelines and the patient medical history that can be reached directly from the displayed patient medical path, wherein the abbreviated representations each display a limited list of data offered within the treatment guidelines and/or the patient medical history, each of the data in the list being selectable to launch the treatment guidelines and/or the patient medical history and enable the selected data to be seen, and wherein the abbreviated representations are displayed while in an unlaunched state. In a first example of the system, segments of the treatment guidelines are selected for the abbreviated representations according to nodes of a treatment path provided in the treatment guidelines, and wherein the nodes are junctions along the treatment path where a continuation of the treatment path is determined based on clinical markers located in the patient medical history. In a second example of the system, optionally including the first example, the patient medical path is displayed based on input entered into a guideline information collection form, and wherein the input entered into the guideline information collection form includes one or more of node name, node type, node display constraints, node entry and exit markers, and node ordering constraints. In a third example of the system, optionally including one or both of the first and second examples, the input entered into the guideline information collection form is stored at a guideline database and used to generate guideline selection rules for a guideline selection module, node traversal rules for a node exit time point detection module, and node ordering rules for an ordering constraint enforcement module, and wherein the said modules include algorithms for displaying the patient medical path. In a fourth example of the system, optionally including one or more or each of the first through third examples, the patient medical path is displayed based on identification of one or more primary clinical findings and tracking a treatment path for each of the one or more primary clinical findings independently. In a fifth example of the system, optionally including one or more or each of the first through fourth examples, the abbreviated representations are user-interactive graphical representations, and wherein the abbreviated representations are in a launched state when a user engages the abbreviated representations by interacting with the abbreviated representations with a cursor.

The disclosure also provides support for a method, comprising: providing a user-interactive electronic form for a user to input rules for displaying a treatment path for a patient, automatically acquiring, via a processor, health records of the patient and treatment guidelines corresponding to a condition of the patient, based on the input rules, and identifying clinical markers from the health records, matching the clinical markers to relevant sections of the treatment guidelines according to implementation of the input rules by the processor, and displaying, at a graphical user interface of a display device in real-time, the relevant sections of the treatment guidelines overlaid with summaries of the health records, the summaries correlated to the relevant sections based on the clinical markers, to provide treatment recommendations based on a path of the treatment guidelines. In a first example of the method, displaying the relevant sections of the treatment guidelines overlaid with the summaries of the health records includes presenting the relevant sections of the treatment guidelines as graphical representations at the graphical user interface. In a second example of the method, optionally including the first example, displaying the relevant sections of the treatment guidelines with the summaries includes ordering the summaries according to a sequence of the treatment guidelines and aligning a corresponding summary of the summaries with each of the graphical representations. In a third example of the method, optionally including one or both of the first and second examples, matching the clinical markers to the relevant sections of the treatment guidelines includes identifying nodes of the treatment guidelines based on a set of guideline selection rules of the input rules. In a fourth example of the method, optionally including one or more or each of the first through third examples, matching the clinical markers to the relevant sections of the treatment guidelines further includes comparing entry markers and exit markers for each of the nodes with information from the clinical markers, and wherein the entry markers and the exit markers are defined by a set of node traversal rules of the input rules. In a fifth example of the method, optionally including one or more or each of the first through fourth examples, comparing the exit markers for each of the nodes with the information from the clinical markers includes determining if the information from the clinical markers satisfies the exit markers of a first node of the nodes and wherein entry markers for a subsequent, second node are compared with the information from the clinical markers to determine entry to the second node when the exit markers of the first node are satisfied by the information from the clinical markers. In a sixth example of the method, optionally including one or more or each of the first through fifth examples, the method further comprises: generating a clinical marker list at a clinical marker detection module by identifying target clinical markers in the treatment guidelines and/or the health records, and wherein identifying the clinical markers from the health records includes locating the target clinical markers in corresponding records of the health records to the clinical marker list and selecting the corresponding records from the health records for display. In a seventh example of the method, optionally including one or more or each of the first through sixth examples, locating the target clinical markers in the corresponding records includes utilizing natural language processing to analyze the corresponding records, and wherein utilizing the natural language processing includes applying one or more of entity recognition, assertion recognition, relation recognition, ontology linking, and clinical marker recognition to the corresponding records. In an eighth example of the method, optionally including one or more or each of the first through seventh examples, identifying the clinical markers from the health records includes identifying one or more of clinical findings, procedures, and biomarkers from the health records. In a ninth example of the method, optionally including one or more or each of the first through eighth examples, the method further comprises: generating the summaries based on one or more of a clinical marker list, filter rules, and summarization templates. In a tenth example of the method, optionally including one or more or each of the first through ninth examples, the treatment guidelines are one or more documents providing standardized treatment recommendations for a health diagnosis, and wherein the relevant sections of the treatment guidelines are updated according to updates to the standardized treatment recommendations.

The disclosure also provides support for a system for displaying a patient journey, comprising: a guideline information collection form providing parameters for displaying a treatment path for a patient stored at a guideline database, treatment guidelines corresponding to the treatment path for the patient stored at a memory of a processor, and a set of modules also stored at the memory of the processor and implemented at the processor, the set of modules including with executable instructions that, when executed, cause the processor to: process the parameters from the guideline information collection form, identify the treatment path from the treatment guidelines based on the parameters, and display the treatment path at a display device along with corresponding summaries from medical records of the patient, the summaries correlated to the treatment path based on common clinical markers. In a first example of the system, the summaries are generated based on input received at a summary rules collection, and stored at a summary rules database, and wherein the input is used to create filter rules for a clinical marker filter module of the set of modules and summarization templates for a summary generation module of the set of modules. In a second example of the system, optionally including the first example, the clinical marker filter module is configured to filter clinical markers received from a clinical marker detection module of the set of modules, and wherein the summary generation module is configured to complete summarization templates with values received from the clinical marker filter module to create the summaries.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A computing system, comprising:
a display screen configured to display a patient medical path listing one or more of treatment guidelines and patient medical history, and additionally being configured to display abbreviated representations of at least one of the treatment guidelines and the patient medical history that can be reached directly from the displayed patient medical path,
wherein the abbreviated representations each display a limited list of data offered within the treatment guidelines and/or the patient medical history, each of the data in the list being selectable to launch the treatment guidelines and/or the patient medical history and enable the selected data to be seen, and
wherein the abbreviated representations are displayed while in an unlaunched state.

2. The computing system of claim 1, wherein segments of the treatment guidelines are selected for the abbreviated representations according to nodes of a treatment path provided in the treatment guidelines, and wherein the nodes are junctions along the treatment path where a continuation of the treatment path is determined based on clinical markers located in the patient medical history.

3. The computing system of claim 1, wherein the patient medical path is displayed based on input entered into a guideline information collection form, and wherein the input entered into the guideline information collection form includes one or more of node name, node type, node display constraints, node entry and exit markers, and node ordering constraints.

4. The computing system of claim 3, wherein the input entered into the guideline information collection form is stored at a guideline database and used to generate guideline selection rules for a guideline selection module, node traversal rules for a node exit time point detection module, and node ordering rules for an ordering constraint enforcement module, and wherein the said modules include algorithms for displaying the patient medical path.

5. The computing system of claim 1, wherein the patient medical path is displayed based on identification of one or more primary clinical findings and tracking a treatment path for each of the one or more primary clinical findings independently.

6. The computing system of claim 1, wherein the abbreviated representations are user-interactive graphical representations, and wherein the abbreviated representations are in a launched state when a user engages the abbreviated representations by interacting with the abbreviated representations with a cursor.

7. A method, comprising:
providing a user-interactive electronic form for a user to input rules for displaying a treatment path for a patient;
automatically acquiring, via a processor, health records of the patient and treatment guidelines corresponding to a condition of the patient, based on the input rules, and identifying clinical markers from the health records;
matching the clinical markers to relevant sections of the treatment guidelines according to implementation of the input rules by the processor; and
displaying, at a graphical user interface of a display device in real-time, the relevant sections of the treatment guidelines overlaid with summaries of the health records, the summaries correlated to the relevant sections based on the clinical markers, to provide treatment recommendations based on a path of the treatment guidelines.

8. The method of claim 7, wherein displaying the relevant sections of the treatment guidelines overlaid with the summaries of the health records includes presenting the relevant sections of the treatment guidelines as graphical representations at the graphical user interface.

9. The method of claim 8, wherein displaying the relevant sections of the treatment guidelines with the summaries includes ordering the summaries according to a sequence of the treatment guidelines and aligning a corresponding summary of the summaries with each of the graphical representations.

10. The method of claim 7, wherein matching the clinical markers to the relevant sections of the treatment guidelines includes identifying nodes of the treatment guidelines based on a set of guideline selection rules of the input rules.

11. The method of claim 10, wherein matching the clinical markers to the relevant sections of the treatment guidelines further includes comparing entry markers and exit markers for each of the nodes with information from the clinical markers, and wherein the entry markers and the exit markers are defined by a set of node traversal rules of the input rules.

12. The method of claim 11, wherein comparing the exit markers for each of the nodes with the information from the clinical markers includes determining if the information from the clinical markers satisfies the exit markers of a first node of the nodes and wherein entry markers for a subsequent, second node are compared with the information from the clinical markers to determine entry to the second node when the exit markers of the first node are satisfied by the information from the clinical markers.

13. The method of claim 7, further comprising generating a clinical marker list at a clinical marker detection module by identifying target clinical markers in the treatment guidelines and/or the health records, and wherein identifying the clinical markers from the health records includes locating the target clinical markers in corresponding records of the health records to the clinical marker list and selecting the corresponding records from the health records for display.

14. The method of claim 13, wherein locating the target clinical markers in the corresponding records includes utilizing natural language processing to analyze the corresponding records, and wherein utilizing the natural language processing includes applying one or more of entity recognition, assertion recognition, relation recognition, ontology linking, and clinical marker recognition to the corresponding records.

15. The method of claim 7, wherein identifying the clinical markers from the health records includes identifying one or more of clinical findings, procedures, and biomarkers from the health records.

16. The method of claim 7, further comprising generating the summaries based on one or more of a clinical marker list, filter rules, and summarization templates.

17. The method of claim 7, wherein the treatment guidelines are one or more documents providing standardized treatment recommendations for a health diagnosis, and wherein the relevant sections of the treatment guidelines are updated according to updates to the standardized treatment recommendations.

18. A system for displaying a patient journey, comprising:
a guideline information collection form providing parameters for displaying a treatment path for a patient stored at a guideline database;
treatment guidelines corresponding to the treatment path for the patient stored at a memory of a processor; and
a set of modules also stored at the memory of the processor and implemented at the processor, the set of modules including with executable instructions that, when executed, cause the processor to:
process the parameters from the guideline information collection form;
identify the treatment path from the treatment guidelines based on the parameters; and
display the treatment path at a display device along with corresponding summaries from medical records of the patient, the summaries correlated to the treatment path based on common clinical markers.

19. The system of claim 18, wherein the summaries are generated based on input received at a summary rules collection, and stored at a summary rules database, and wherein the input is used to create filter rules for a clinical marker filter module of the set of modules and summarization templates for a summary generation module of the set of modules.

20. The system of claim 19, wherein the clinical marker filter module is configured to filter clinical markers received from a clinical marker detection module of the set of modules, and wherein the summary generation module is configured to complete summarization templates with values received from the clinical marker filter module to create the summaries.
